(19) United States
(12) Patent Application Publication
(10) Pub. No.: US 2024/0321433 A1
(43) Pub. Date: Sep. 26, 2024

(54) MEDICAL IMAGE DISPLAY APPARATUS, MEDICAL IMAGE DISPLAY SYSTEM, MEDICAL IMAGE DISPLAY METHOD, AND RECORDING MEDIUM

(71) Applicant: KONICA MINOLTA, INC., Tokyo (JP)

(72) Inventors: Hidekazu TAKAHASHI, Osaka (JP); Hisashi WADA, Tokyo (JP); Ken SATOU, Tokyo (JP); Kohei IKEDA, Tokyo (JP); Ryuta OGAWA, Osaka (JP); Seiji NOMURA, Tokyo (JP)

(73) Assignee: KONICA MINOLTA, INC., Tokyo (JP)

(21) Appl. No.: 18/596,666

(22) Filed: Mar. 6, 2024

(30) Foreign Application Priority Data
Mar. 24, 2023 (JP) 2023-048405

(51) Int. Cl. G16H 30/40; G16H 10/60; G16H 30/20

(52) U.S. Cl. CPC G16H 30/40; G16H 10/60; G16H 30/20

(57) ABSTRACT

A medical image display apparatus includes a display that is capable of displaying a first medical image acquired from a first server and a second medical image acquired from a second server. The display displays information indicating that the first medical image and the second medical image coexist.

### BACKGROUND OF THE INVENTION

### Technical Field

The present invention relates to a medical image display apparatus, a medical image display system, a medical image display method, and a recording medium.

### Description of Related Art

In recent years, in medical facilities such as hospitals and clinics, differentiation of functions has advanced. In addition, a series of responses (examination, diagnosis, treatment, and the like) to one disease of a patient has been spread over a plurality of facilities.

In addition, with the development of cloud systems, a technology for collecting and managing data of a plurality of facilities in the cloud has advanced.

However, at present, identification information (patient ID or the like) for specifying a patient is different from facility to facility, and hence it is difficult to collectively grasp data of a patient(s) existing across a plurality of facilities.

Therefore, for example, there is a movement to manage a patient with common identification information such as a region ID unique in a region, My Number (R), or a medical ID.

However, it is difficult to say that such common identification information generally spreads. Generally, a patient is still managed using unique identification information (patient ID or the like) on a facility-by-facility basis.

Then, for example, Japanese Unexamined Patent Publication No. 2017-215883 discloses a medical image display apparatus for identifying the identical patient by the so-called name-based aggregation technology. The purpose of this is to enable viewing of data such as medical images of a patient managed across facilities or regions.

In the name-based aggregation technology, first, a search for a relevant patient similar to a search target patient is performed. Next, patient information corresponding to the search target patient and patient information corresponding to the relevant patient are displayed in the form of a list on a display means so that they can be compared with one another to find difference therebetween.

In the medical image display apparatus of Japanese Unexamined Patent Publication No. 2017-215883, a name-based aggregation list is displayed, and the identical patient is specified in advance using the name-based aggregation list.

### SUMMARY OF THE INVENTION

However, it is not guaranteed that the specified patient is identical with the search target patient.

Therefore, when the examination information of the search target patient is displayed on the same viewer, there is a risk that the user does not notice that the examination information of the patient is the examination information of a non-identical patient.

An object of the present invention is to reduce the risk of erroneously recognizing patient information on a non-identical patient as patient information on an identical patient.

To achieve at least one of the abovementioned objects, according to an aspect of the present invention, a medical image display apparatus reflecting one aspect of the present invention includes a display that is capable of displaying a first medical image acquired from a first server and a second medical image acquired from a second server,

wherein the display displays information indicating that the first medical image and the second medical image coexist.

According to an aspect of the present invention, a non-transitory computer-readable recording medium reflecting one aspect of the present invention stores a program that causes a hardware processor of a medical image display apparatus to:

cause a display to display a first medical image acquired from a first server and a second medical image acquired from a second server; and

cause the display to display information indicating that the first medical image and the second medical image coexist.

According to an aspect of the present invention, a medical image display system reflecting one aspect of the present invention includes a display that is capable of displaying a first medical image acquired from a first server and a second medical image acquired from a second server,

wherein the display displays information indicating that the first medical image and the second medical image coexist.

According to an aspect of the present invention, a medical image display method reflecting one aspect of the present invention includes:

causing a display to display a first medical image acquired from a first server and a second medical image acquired from a second server; and

causing the display to display information indicating that the first medical image and the second medical image coexist.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention, wherein:

FIG. 1 is a block diagram illustrating a configuration of a medical image display system;

FIG. 2 is a block diagram illustrating a configuration of a medical image display apparatus;

FIG. 3 is a flowchart illustrating a medical image display process executed by the medical image display apparatus;

FIG. 4 is a diagram illustrating a work list in which a list of examinations satisfying a predetermined search condition is displayed;

FIG. 5 is a diagram showing a work list after examination selection;

FIG. 6 is a diagram illustrating a work list in a case where a relevant patient exists;

FIG. 7 is a diagram illustrating a work list after an operation on existence information;

FIG. 8 is a diagram illustrating an examination list of relevant patients; and

FIG. 9 is a diagram illustrating a viewer in a case where medical images of examinations performed in different medical facilities are displayed.

### DETAILED DESCRIPTION

Hereinafter, one or more embodiments of the present invention will be described with reference to the drawings. However, the scope of the invention is not limited to the disclosed embodiments or illustrated examples.

### [Medical Image Display System]

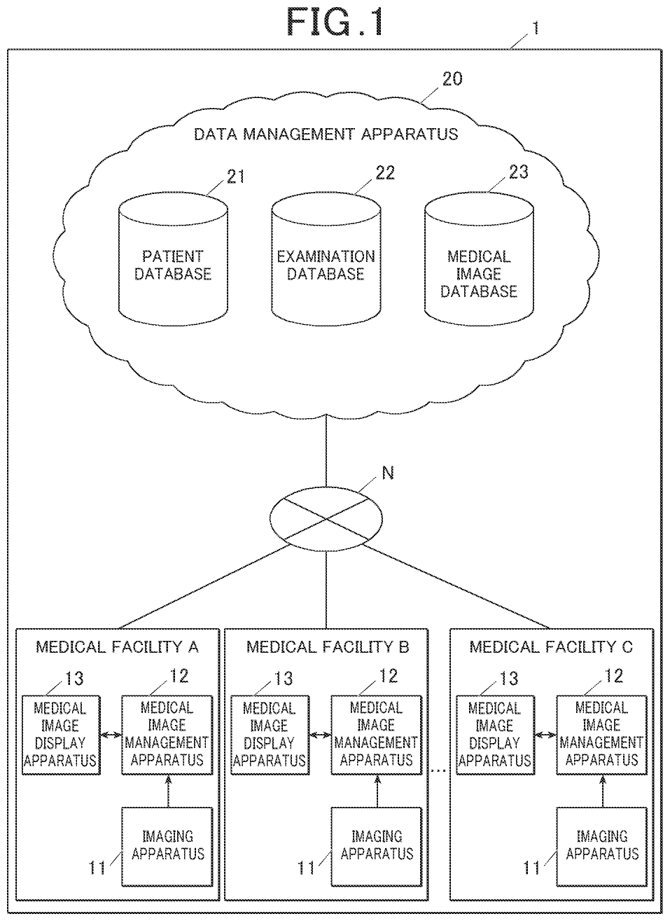

FIG. 1 shows a configuration of a medical image display system 1. The medical image display system 1 includes imaging apparatuses 11, medical image management apparatuses 12 and medical image display apparatuses 13 in medical facilities A, B, . . . and X. The medical image display system 1 further includes a data management apparatus 20. The apparatuses constituting the medical image display system 1 are connected to each other via a communication network N such as a local area network (LAN), a wide area network (WAN), or the Internet. Each apparatus constituting the medical image display system 1 conforms to the HL7 (Health Level Seven) or DICOM (Digital Image and Communications in Medicine) standard. Therefore, communication between the apparatuses is performed in accordance with HL7 or DICOM.

### [Imaging Apparatus]

The imaging apparatus 11 is an image generating apparatus such as an X-ray imaging apparatus (DR, CR), an ultrasonic diagnostic apparatus (US), a CT, or an MRI. The imaging apparatus 11 generates a medical image as medical information by imaging an examination target site of a patient as a subject based on examination order information transmitted from a radiology information system (RIS) (not illustrated) or the like.

In the medical image generated by the imaging apparatus 11, supplementary information (patient information, examination information, image ID, and the like) is written in, for example, a header of an image file in accordance with the DICOM standard. The medical image appended with the supplementary information is transmitted to the medical image management apparatus 12 via a console or the like (not illustrated). Note that a configuration may be adopted in which a medical image is transmitted from the console to an image analysis apparatus, and an analysis image on which predetermined analysis process has been performed is transmitted to the medical image management apparatus 12.

### [Medical Image Management Apparatus]

The medical image management apparatus 12 is, for example, a server constituting a picture archiving and communication system (PACS). Upon receiving the medical image from the imaging apparatus 11, the medical image management apparatus 12 stores the medical image in a database in association with patient information, examination information, the image ID of the medical image, the facility ID of the medical facility, and the like. Then, the medical image management apparatus 12 transmits the stored copy of the medical image to the data management apparatus 20.

### [Medical Image Display Apparatus]

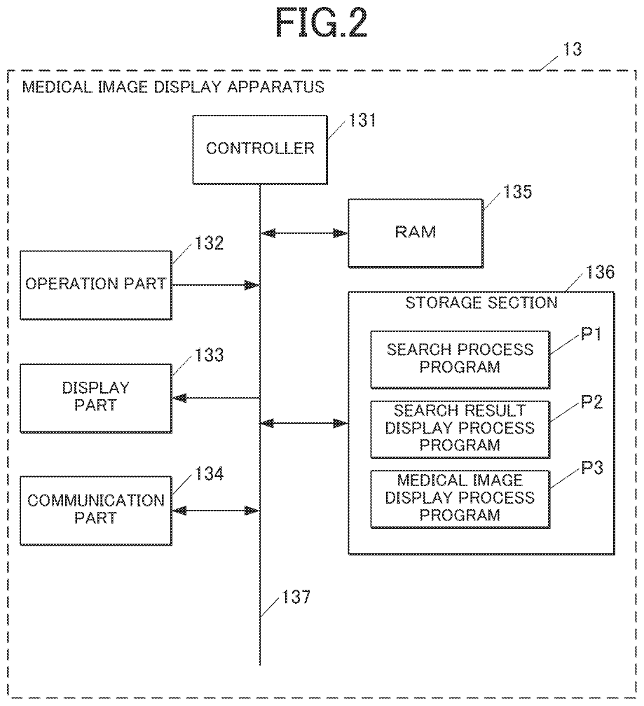

The medical image display apparatus 13 is an apparatus that displays a medical image captured in a specified examination. FIG. 2 is a block diagram illustrating a functional configuration of the medical image display apparatus 13. The medical image display apparatus 13 includes a controller 131 (hardware processor), an operation part 132, a display part 133 (display), a communication part 134, a RAM (Random Access Memory) 135, and a storage section 136. The respective units of the medical image display apparatus 13 are connected by a bus 137.

### (Controller)

The controller 131 includes a central processing unit (CPU) and the like. The controller 131 reads a system program and various process programs stored in the storage section 136, and develops them in a work area formed in the RAM 135. The controller 131 controls each unit in accordance with the program.

The controller 131 functions as a hardware processor that causes the display part to display a first medical image acquired from a first server and a second medical image acquired from a second server.

### (Operation Part)

The operation part 132 includes a keyboard having cursor keys, character input keys, various function keys, and the like, and a pointing device such as a mouse. The operation part 132 outputs, to the controller 131, an instruction signal input by a key operation on the keyboard or a mouse operation. The operation part 132 may include a touch screen formed so as to cover the display part 133.

### (Display Part)

The display part 133 is configured by a monitor such as a liquid crystal display (LCD). The display part 133 displays various screens and the like in accordance with an instruction of a display signal input from the controller 131.

The display part 133 functions as a display part capable of displaying the first medical image acquired from the first server and the second medical image acquired from the second server. The first server and the second server will be described later.

The first server is a server that manages medical images, and the second server is a server that manages medical images other than the first server. For example, the first server refers to a server of the medical image management apparatus 12 in the medical facility A. For example, the second server refers to a server of the medical image management apparatus 12 in the medical facility B, the medical facility C or the like other than the medical facility A, or a server of the data management apparatus 20.

Further, in the data management apparatus 20 which is a virtual apparatus provided in a cloud environment, a server which manages medical images of the medical facility A may be the first server. On that basis, a server that manages medical images other than the first server may be the second server.

Although the example has been described above with the medical facility A as the base, the medical facility B or the like may be the base.

Note that the first server and the second server correspond to the respective medical facilities of the plurality of medical facilities, and therefore can be said to belong to different medical facilities.

### (Communication Part)

The communication part 134 includes a LAN adapter, a router, and a terminal adapter (TA). The communication part 134 transmits and receives data to and from an external apparatus connected to a communication network.

### (RAM)

The RAM 135 forms a work area. The work area temporarily stores various programs read from the storage section 136, input or output data, parameters, and the like in various processes executed and controlled by the controller 131.

### (Storage Section)

The storage section 136 is formed with a hard disk, a semiconductor nonvolatile memory, or the like. The storage section 136 stores a system program and various process programs to be executed by the controller 131 and data necessary for executing these programs. The storage section 136 stores, for example, a search process program P1, a search result display process program P2, and a medical image display process program P3.

### {Search Process Program}

The search process program P1 is a program for executing an examination search process and a relevant patient search process. The examination search process is a process of searching for and extracting an examination that matches a predetermined search condition from the medical image management apparatus 12. The relevant patient search process is a process of searching for and extracting a second patient from the patient database 21. Here, the second patient is a relevant patient having individual identification information matching the individual identification information of a first patient to be a target.

### {Search Result Display Process Program}

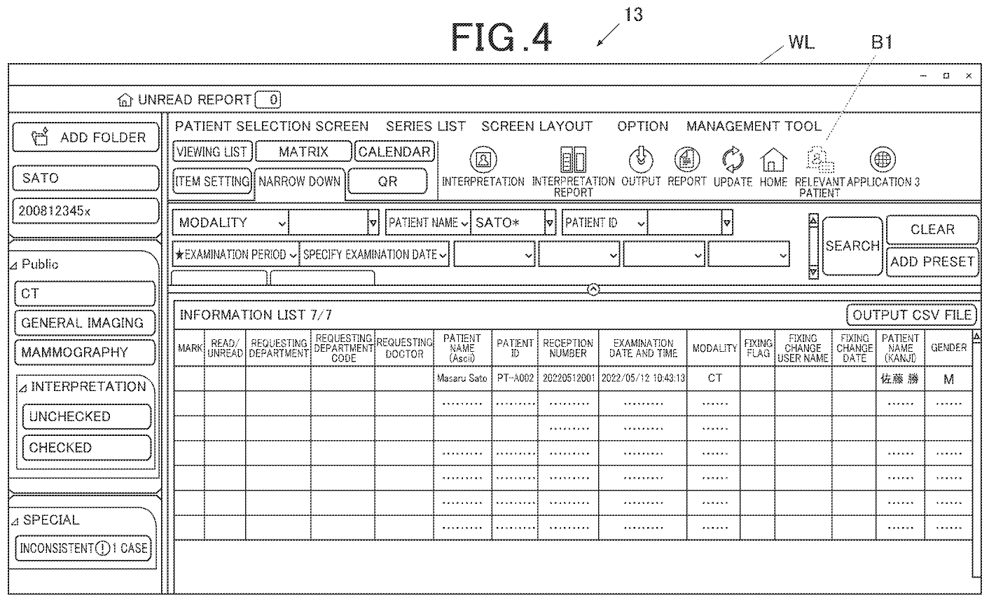

The search result display process program P2 is a program for executing a patient search result display process and an examination search result display process. The examination search result display process is a process of displaying, in the work list WL (see FIG. 4), a list of examinations extracted by execution of the search process program P1. The relevant patient search result display process is a process of displaying, in the work list WL, a list of relevant patients extracted by execution of the search process program P1.

### {Medical Image Display Process Program}

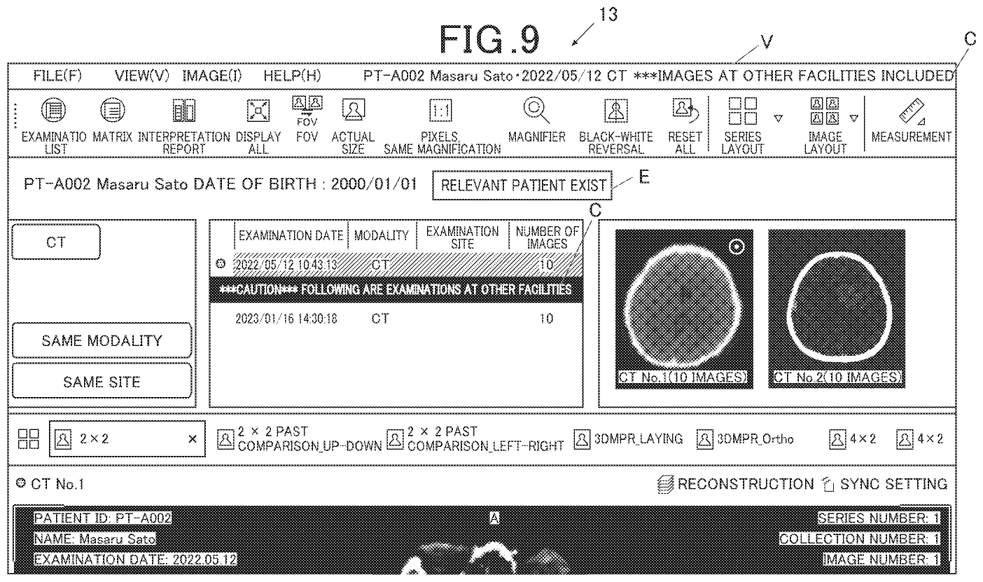

The medical image display process program P3 is a program for executing a medical image display process. The medical image display process is a process of acquiring a medical image of a specified examination from the list of examinations displayed by the examination search result display process, from the medical image management apparatus 12 or the medical image database 23, and displaying the medical image on a viewer V (see FIG. 9).

### [Data Management Apparatus]

Returning to FIG. 1, the data management apparatus 20 is, for example, a virtual apparatus provided in a cloud environment. The data management apparatus 20 is connected to the medical image management apparatuses 12 and the medical image display apparatuses 13 in a plurality of medical facilities. The data management apparatus 20 includes a patient database 21, an examination database 22, a medical image database 23, and the like corresponding to each medical facility. The data management apparatus 20 stores the medical image received from each medical image management apparatus 12 in a corresponding medical image database 23. The data management apparatus 20 transmits, based on the communication from each medical image display apparatus 13, the data of each database to the medical image display apparatus 13.

### {Patient Database}

The patient database 21 stores individual identification information of each patient in each medical facility. The individual identification information includes a plurality of items (elements). The individual identification information includes, for example, patient information such as a patient ID, the patient's name (kanji, kana, alphabet), date of birth (age), gender, and a medical facility.

Note that the individual identification information may be separate (different) for each medical facility.

Note that in the patient database 21, each patient information may be associated with medical information such as medical record information and vital information. The medical information is generated in the same medical facility as the patient information associated with the medical information.

### {Examination Database}

The examination database 22 stores examination identification information on each examination performed at each medical facility. The examination identification information includes a plurality of items (elements). The examination identification information includes, for example, an examination ID, a patient's name (kanji, kana, and alphabet), date of birth, gender, a medical facility, an examination date, a modality, and an imaging site. The items of the examination identification information include a path for the medical image of the patient captured in the examination and saved in the medical image database 23.

### {Medical Image Database}

The medical image database 23 stores medical images captured by the imaging apparatus 11 and received from the medical image management apparatus 12 in examinations performed in each of a plurality of medical facilities. In the medical image database 23, supplementary information is stored in association with each medical image. The supplementary information includes patient information, examination information, series information, image information, and the like.

The patient information is information on a patient. The patient information includes a patient ID, a patient name, date of birth, age, gender, and the like.

The examination information is information on an examination. The examination information includes an examination instance UID, an examination date and time, and an examination description.

The series information is information on a series. The series information includes a modality (CT, MRI, DR, CR, US, or the like), a series instance UID, an examination site, a series number, a series description, and the like. The series instance UID is a unique ID for identifying a series. The series number is a number indicating the order of the series in the same examination, and is used as the display order or the like in some cases.

The image information is information on a medical image. The image information includes an image number, an image generation date and time, an imaging direction, an imaging start position, an image type, a file path, and the like. The image number is a number indicating the ordinal number of an image in the same series. The imaging start position is coordinates indicating a position at which an examination (scanning) is started in an examination such as CT or MRI. The image type is information indicating whether the image is a slice image or a scout image in an examination of CT, MRI, or the like. The file path is information indicating a storage location of the medical image in the medical image database 23.

### [Medical Image Display Process]

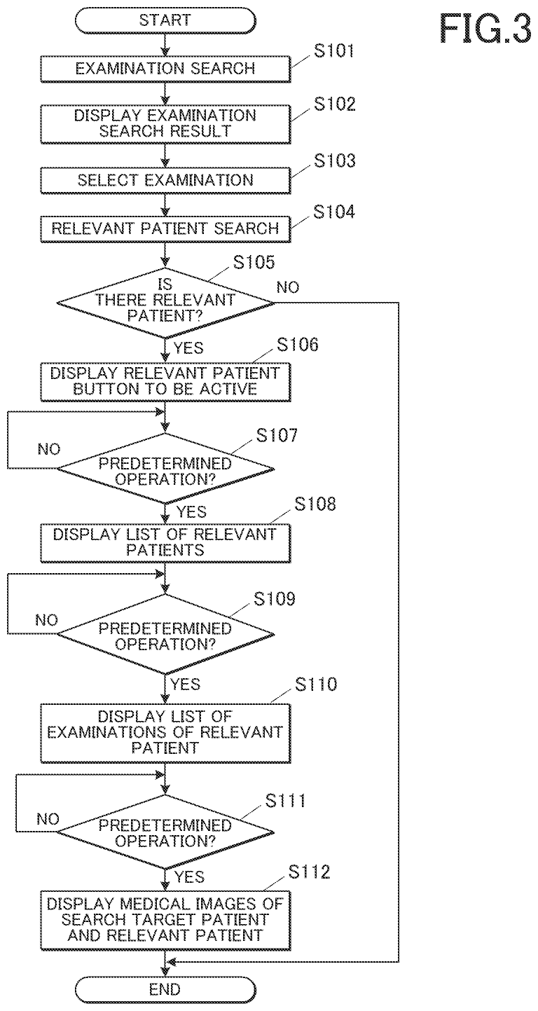

Next, the operation of the medical image display apparatus 13 will be described. FIG. 3 is a flowchart illustrating the medical image display process executed by the medical image display apparatus 13. The medical image display process is implemented by software process or the like in cooperation with the controller 131 and a program stored in the storage section 136. The medical image display process is started when the user activates the work list WL.

First, the user inputs a predetermined search condition(s) and operates a search button by operating the operation part 132. Then, the controller 131 executes the examination search process by the search process program P1 (Step S101). Next, the controller 131 acquires, from the medical image management apparatus 12, examination data of the own facility that satisfies/satisfy the predetermined search condition.

The controller 131 executes the examination search result display process by the search result display process program P2 (Step S102). Through the examination search result display process, the controller 131 causes the work list WL to display a list of examinations satisfying the predetermined search condition. FIG. 4 illustrates an example of the work list WL in which a list of examinations satisfying the predetermined search condition is displayed. In FIG. 4, the controller 131 displays an examination list for the patient name of “Sato”, which is the predetermined search condition, in the work list WL.

In this step, the examination information is in a non-selected state, and a relevant patient button B1 is in an inactive state. The relevant patient button B1 will be described later.

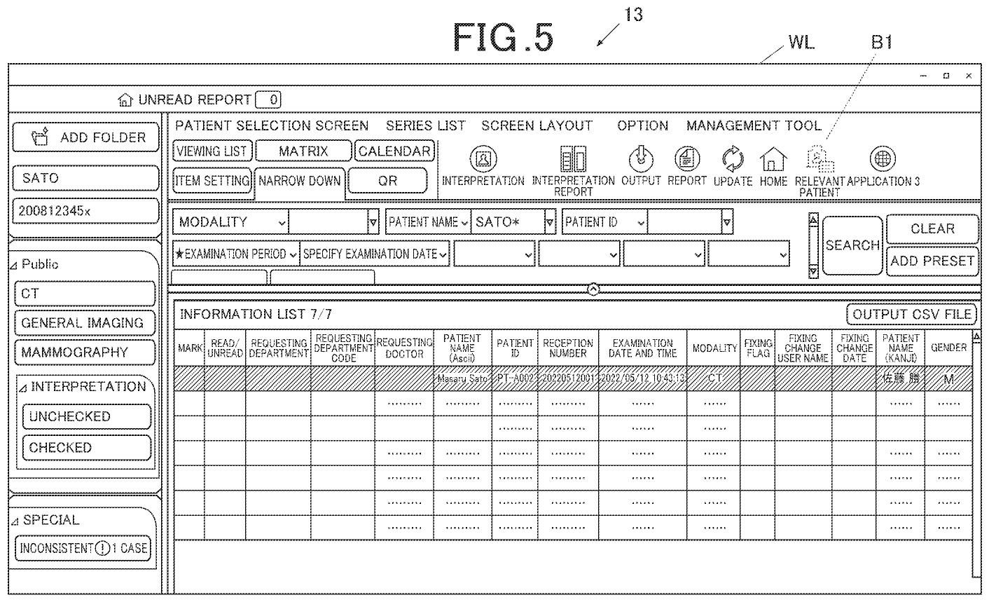

The controller 131 receives specification of a first examination by a predetermined operation of the user (Step S103). For example, as illustrated in FIG. 5, when the row of “Masaru Sato” whose patient ID is PT-A002 is clicked once, the color of the row changes and display thereof is in a selected state.

Returning to FIG. 3, the controller 131 that has received the specification of the first examination executes the relevant patient search process by the search process program P1 (Step S104). Then, the controller 131 acquires data of a second patient who is a relevant patient having individual identification information similar to that of the first patient of the specified first examination, from the patient database 21.

In the present embodiment, the controller 131 executes the relevant patient search process by the so-called name-based aggregation search. The name-based aggregation search is a search method of searching for a relevant patient(s) on the basis of first individual identification information identifying the first patient. Here, the relevant patient is a second patient having second individual identification information which is individual identification information similar to the first individual identification information.

Here, the relevant patient search process based on the name-based aggregation search by the controller 131 will be described. The name-based aggregation search includes, for example, searching for a relevant patient stored in the patient database 21 of a first medical facility (for example, the medical facility A) using the patient information of the first patient stored in the patient database 21 of the first medical facility. The name-based aggregation search includes searching for a relevant patient stored in the patient database 21 of a second medical facility (for example, the medical facility B) using the patient information of the first patient stored in the patient database 21 of the first medical facility. Of course, the relevant patient who the name-based aggregation search searches for may be the same person as the search target patient.

In the name-based aggregation search, for example, five items of the individual identification information that are, for example, the name (in kanji, kana and alphabet), the date of birth and the gender of the patient are used as name-based aggregation conditions. A search condition(s) as to which item of the individual identification information is used as a name-based aggregation condition(s) is stored in the storage section 136 in advance.

First, the controller 131 compares the individual identification information of the search target patient with that of the other patients on an item-by-item basis. Then, the controller 131 adds a penalty value of an unmatched item to a reference value “1”, and calculates the similarity between the patients by taking the reciprocal of the addition result.

For example, it is assumed that patients match in name in kana, name in alphabet and gender, but do not match in name in kanji and date of birth. Further, it is assumed that the penalty values are set at “5” for name in kanji, “1” for name in kana, “1” for name in alphabet, “3” for date of birth, and “1” for gender, for example. In this case, the similarity is obtained as follows.

Similarity=1/{reference value+penalty value for name in kanji+penalty value for date of birth}

=1/(1+5+3)

=0.11

As another example, it is assumed that patients match in name in kanji, date of birth and gender, but do not match in name in kana and name in alphabet. It is assumed that the penalty values are set in the same manner as described above. In this case, the similarity is obtained as follows.

Similarity=1/{reference value+penalty value for name in kana+penalty value for name in alphabet}

=1/(1+1+1)

=0.33

As the name-based aggregation search, the controller 131 calculates the similarity between the search target patient and the other patients as described above, and extracts another patient(s) whose similarity is equal to or greater than a predetermined value as a relevant patient(s). Thus, the controller 131 functions as a determination section that determines the presence or absence of a relevant patient satisfying the name-based aggregation conditions. Further, the controller 131 functions as a calculation section that calculates the similarity between the first patient and the relevant patient. The patient information of the relevant patient extracted by the name-based aggregation search is stored in the RAM 135.

The controller 131 may set items other than the above-described five items as the name-based aggregation conditions. For example, the modality of the imaging apparatus may be included in the name-based aggregation conditions. Alternatively or additionally, the controller 131 may include the imaging site of the medical image in the name-based aggregation conditions. Furthermore, the items to be included in the name-based aggregation conditions and the method of determining the presence or absence of a relevant patient in the name-based aggregation search may be appropriately changeable by user operations. Thus, the controller 131 functions as a name-based aggregation condition setting section that sets name-based aggregation conditions in the patient database 21.

The modality of the imaging apparatus and the data related to the imaging site of the medical image may be stored in the examination database 22 as the examination information as described above. Alternatively, the controller 131 may be configured to function as a specifying section that specifies these pieces of information from the medical images stored in the medical image database 23 with the viewer V or the work list WL.

The controller 131 determines the presence or absence of a relevant patient in the patient database 21 (Step S105). If the relevant patient is not present in the patient database 21 (Step S105; No), the medical image display process ends.

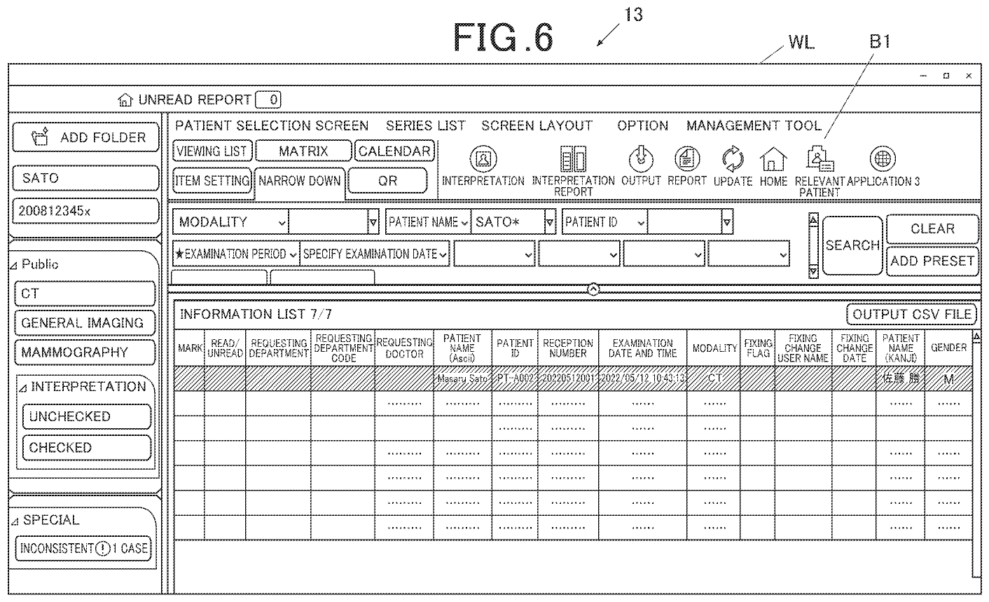

On the other hand, if the relevant patient exists in the patient database 21 (Step S105; Yes), the controller 131 changes the relevant patient button B1 to an active state (Step S106). As illustrated in FIG. 6, the relevant patient button B1 changes to the active state if a relevant patient exists.

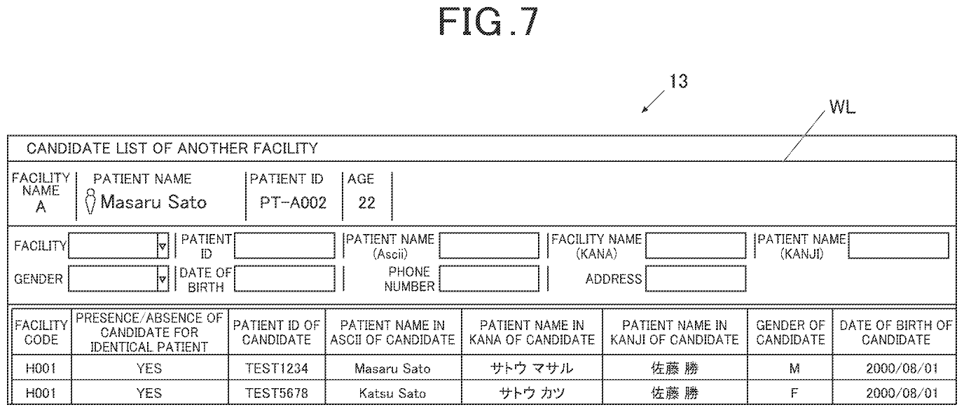

The controller 131 receives a predetermined operation (Step S107). For example, in the present embodiment, the predetermined operation is a click on the relevant patient button B1. The relevant patient button B1 is a button for executing the search result display process program P2. When receiving the predetermined operation (Step S107; Yes), the controller 131 executes the search result display process program P2. Next, as illustrated in FIG. 7, the controller 131 causes a list of pieces of the patient information of the relevant patients stored in the RAM 135 to be displayed in the work list WL (Step S108).

The controller 131 receives a predetermined operation on the work list WL (Step S109). In the present embodiment, the predetermined operation is, for example, a double click for specifying one relevant patient.

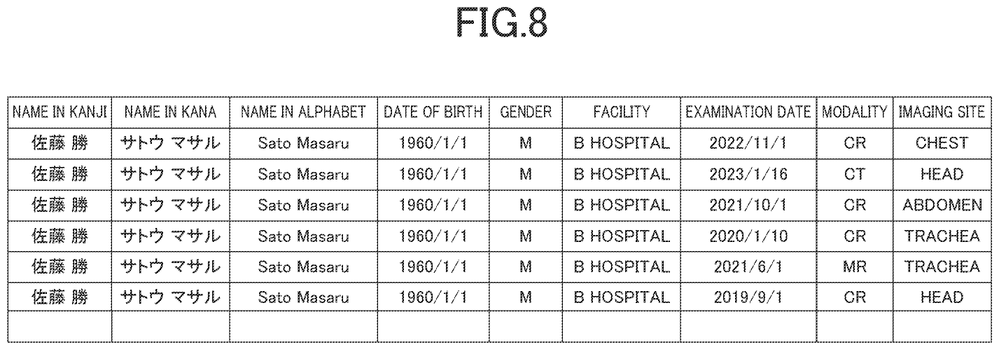

When receiving the predetermined operation on the work list WL (Step S109; Yes), the controller 131 acquires the examination list for the one relevant patient from the examination database 22 with the search process program P1. FIG. 8 shows an example of the examination list. Next, the controller 131 executes the search result display process program P2 to display the examination list of the specified one relevant patient in the work list WL (Step S110).

The controller 131 receives a predetermined operation on the work list WL (Step S111). In the present embodiment, the predetermined operation is, for example, a double click for specifying the second examination.

When receiving the predetermined operation on the work list WL (Step S111; Yes), the controller 131 executes the medical image display process program P3. Next, the controller 131 causes the viewer V to display the medical image of the first examination of the search target patient and the medical image of the second examination of the relevant patient specified in Step S111 together (Step S112; display step).

A case in which the examination specified in Step S111 is an examination performed in a medical facility different from that of the examination specified in Step S103 will be described. In this case, it is preferable to display a warning C in Step S112. The display is exemplified in FIG. 9. With the above configuration, it is possible to prevent the user from not noticing that the search target patient and the relevant patient are different patients. The warning C is information indicating that the first medical image acquired from the first server and the second medical image acquired from the second server coexist.

A case where the examination specified in Step S111 is an examination performed in a facility different from that of the examination selected in Step S103 will be described. In this case, in Step S111, the controller 131 may display the degree of similarity of the examinations. Thus, the user can grasp the degree of risk that the search target patient and the relevant patient are different patients.

A case where the examination specified in Step S111 is an examination performed in a facility different from that of the examination selected in Step S103 will be described. In this case, in Step S111, instead of the warning C, the controller 131 may change the color or font of characters for displaying the examination specified in Step S111 from that for displaying the examination selected in Step S103. In the example of FIG. 9, the controller 131 changes the color or font of characters in the list of the examination information at the center of the viewer V.

### Advantageous Effects of Present Embodiment

As described above, the medical image display apparatus 13 according to the present embodiment includes the display part 133 capable of displaying the first medical image acquired from the first server and the second medical image acquired from the second server, and the display part 133 displays information indicating that the first medical image and the second medical image coexist. Thus, the user can recognize that they coexist. Therefore, while recognizing that there is a risk that the first medical image and the second medical image are of different patients, the user can check the images. Therefore, the risk of erroneously recognizing the patient information on a non-identical patient as the patient information on an identical patient is reduced.

### OTHER CONFIGURATIONS

Note that the description in the above embodiment is an example of the medical image display apparatus according to the present invention, and the present invention is not limited to this. The detailed configuration and detailed operation of each part constituting the apparatus can also be appropriately changed without departing from the scope of the present invention. Although embodiments of the present invention have been described and illustrated in detail, the disclosed embodiments are made for purposes of illustration and example only and not limitation. The scope of the present invention should be interpreted by terms of the appended claims.

For example, operation of each function of the viewer can be changed by settings, and those settings are saved in the program. The saved settings are read when the viewer is activated and reflected in the viewer, so that the user can refer to the image with the same settings of the viewer as those of the previous time.

By keeping these settings for each user, it is possible to refer to an image with each viewer setting for each user.

On the other hand, there are also cases where a plurality of persons refer to an image with the same viewer settings. In this case, contrivance is made to impose a restriction so that users other than the limited users cannot change the settings of each function, or impose a restriction so that the changed settings cannot be saved in the program. This is to prevent the settings from being changed by the users.

These function restrictions may be imposed on each function or on each user.

Further, for example, although in the above embodiment, if no relevant patient exists, only the medical images of the search target patient are displayed on the viewer V, this is not a limitation. For example, the controller 131 may display no-existence information NE indicating that no relevant patient exists on the viewer V illustrated in FIG. 9. With the above-described configuration, it becomes easier for the user to know that there is no relevant patient although the name-based aggregation search is performed.

Further, in the above description, as an example, the ROM (appropriately changeable) is used as a computer-readable medium of the program according to the present invention, but the present invention is not limited to this example. As the computer-readable medium, a nonvolatile memory such as a flash memory or a portable recording medium such as a CD-ROM can be used. As a medium for providing data of the program according to the present invention via a communication line, a carrier wave is also applied to the present invention.

The entire disclosure of Japanese Patent Application No. 2023-048405 filed on Mar. 24, 2023 is incorporated herein by reference in its entirety.

What is claimed is:

1. A medical image display apparatus comprising a display that is capable of displaying a first medical image acquired from a first server and a second medical image acquired from a second server,
wherein the display displays information indicating that the first medical image and the second medical image coexist.

2. The medical image display apparatus according to claim 1, wherein the display displays the first medical image and the second medical image to be distinguishable from one another.

3. The medical image display apparatus according to claim 1, wherein the first server and the second server belong to different medical facilities.

4. The medical image display apparatus according to claim 1, wherein the first server and the second server store pieces of patient information on a patient with different pieces of individual identification information on the patient.

5. The medical image display apparatus according to claim 1, wherein the second medical image is acquired from the second server by a name-based aggregation search.

6. A non-transitory computer-readable recording medium storing a program that causes a hardware processor of a medical image display apparatus to:
cause a display to display a first medical image acquired from a first server and a second medical image acquired from a second server; and
cause the display to display information indicating that the first medical image and the second medical image coexist.

7. The non-transitory computer-readable recording medium according to claim 6, wherein the program causes the hardware processor to cause the display to display the first medical image and the second medical image to be distinguishable from one another.

8. The non-transitory computer-readable recording medium according to claim 6, wherein the first server and the second server belong to different medical facilities.

9. The non-transitory computer-readable recording medium according to claim 6, wherein the first server and the second server store pieces of patient information on a patient with different pieces of individual identification information on the patient.

10. The non-transitory computer-readable recording medium according to claim 6, wherein the second medical image is acquired from the second server by name-based aggregation.

11. A medical image display system comprising a display that is capable of displaying a first medical image acquired from a first server and a second medical image acquired from a second server,
wherein the display displays information indicating that the first medical image and the second medical image coexist.

12. A medical image display method comprising:
causing a display to display a first medical image acquired from a first server and a second medical image acquired from a second server; and
causing the display to display information indicating that the first medical image and the second medical image coexist.
